(19) United States
(12) Patent Application Publication
(10) Pub. No.: US 2022/0142687 A1
(43) Pub. Date: May 12, 2022

(54) BONE RECONSTRUCTION CONNECTION STRUCTURE AND METHOD BASED ON TENON-AND-MORTISE FIXATION

(71) Applicant: SHANGHAI NINTH PEOPLE'S HOSPITAL, JIAOTONG UNIVERSITY SCHOOL OF MEDICINE, SHANGHAI (CN)

(72) Inventors: JIANNAN LIU, SHANGHAI (CN); CHENPING ZHANG, SHANGHAI (CN)

(73) Assignee: SHANGHAI NINTH PEOPLE'S HOSPITAL, JIAOTONG UNIVERSITY SCHOOL OF MEDICINE, SHANGHAI (CN)

(21) Appl. No.: 17/583,937

(22) Filed: Jan. 25, 2022

Related U.S. Application Data
(63) Continuation-in-part of application No. PCT/CN2020/129708, filed on Nov. 18, 2020.

(30) Foreign Application Priority Data
Jul. 31, 2020 (CN) 202021555008.1

(51) Int. Cl. A61B 17/80

(52) U.S. Cl. CPC A61B 17/8004; A61B 2017/564; A61B 17/8071

(57) ABSTRACT

The present disclosure provides a bone reconstruction connection structure and method based on tenon-and-mortise fixation. The bone reconstruction connection structure based on tenon-and-mortise fixation includes at least two bone segments, and adjacent bone segments are detachably connected through a first connecting part and a second connecting part; the first connecting part includes a first tongue and a first shoulder, the first tongue protrudes from the first shoulder; the second connecting part includes a first slot and a first slot shoulder, and the first slot is retracted relative to the first slot shoulder, and the part of the first tongue protruding from the first shoulder matches the first slot. The bone reconstruction connection structure provided by the present disclosure is reinforced only through its own tenon-and-mortise structure, which can reduce the risk of infection, rejection and even secondary operation caused by implant fixation, and promote the healing of broken bone ends.

### CROSS REFERENCE TO RELATED APPLICATION

This is a continuation-in-part application claiming priority to a PCT International Application No. PCT/CN2020/129708, filed on Nov. 18, 2020, which claims the benefit of priority to Chinese Patent Application No. CN 2020215550081, entitled “Bone Reconstruction Connection Structure Based on Tenon-and-mortise fixation”, filed with CNIPA on Jul. 31, 2020, the disclosure of which is incorporated herein by reference in their entirety.

### TECHNICAL FIELD

The present disclosure belongs to the technical field of medical devices, and relates to a bone reconstruction connection structure and method based on tenon-and-mortise fixation.

### BACKGROUND

Bone defects caused by tumor, trauma, inflammation and other factors will have a very serious impact on the body and mind of patients. At present, vascularized autologous bone transplantation is the gold standard of bone reconstruction. Commonly used vascularized bone flaps often include fibula, iliac bone, scapula and the like. There are mainly two solutions to fix the transplanted bone and the broken end of bone:

(1) Kirschner wire fixation: the internal fixation materials commonly used in orthopedics are used for fixation. The specification of this internal fixation material is generally about 20 cm, and the diameter is 0.5-2 mm. It is mostly used for the fixation of fractures with low stress such as short fractures or avulsion fractures and the like. It is also often used for the fixation of temporary fracture blocks in orthopedic operation. However, Kirschner wires often need to pass through the skin and be exposed on the surface. If the dressing is not changed in time, it is easy to cause wound infection and often needs to be removed by a secondary operation.

(2) Titanium plate rigid internal fixation: the titanium plates made of titanium or titanium alloy are used to fix both ends of the fracture line together with titanium nails, as shown in FIG. 1, so as to stabilize the broken end of bone and to promote bone healing. However, titanium or titanium alloy is used as raw material in titanium plate rigid internal fixation method, and a few people have infection and rejection against titanium metal which need to be removed by a secondary operation; at the same time, the bearing mode of titanium plate does not meet the requirements of mandibular biomechanics.

Therefore, it is necessary to further study and explore the fixation methods in bone reconstruction.

### SUMMARY

The present disclosure provides a bone reconstruction connection structure and method based on tenon-and-mortise fixation to promote the healing of bone broken ends without the help of foreign objects such as fixing plates.

The first aspect of the present disclosure provides a bone reconstruction connection structure based on tenon-and-mortise fixation, including at least two bone segments, and the adjacent bone segments are detachably connected through a first connecting part and a second connecting part; the first connecting part includes a first tongue and a first shoulder, the first tongue protrudes from the first shoulder, the second connecting part includes a first slot and a first slot shoulder, the first slot is retracted relative to the first slot shoulder, and the part of the first tongue protruding from the first shoulder matches the first slot.

Preferably, the bone segments include a first bone segment, a second bone segment and a third bone segment connected in sequence. A first connecting part or a second connecting part arranged at one end of the second bone segment is detachably connected to a second connecting part or a first connecting part arranged at one end of the first bone segment. A first connecting part or a second connecting part arranged at the other end of the second bone segment is detachably connected to a second connecting part or a first connecting part arranged at one end of the third bone segment.

More preferably, a first bending angle of 60-180° is formed between the second bone segment and the first bone segment; a second bending angle of 60-180° is formed between the second bone segment and the third bone segment; the bending direction of the first bone segment relative to the second bone segment is opposite to the bending direction of the third bone segment relative to the second bone segment.

More preferably, the connecting end between the second bone segment and the first bone segment, and the connecting end between the second bone segment and the third bone segment are provided with bone rods, and the bone rods include a first bone rod and a second bone rod. The first bone rod is arranged at the connecting end between the second bone segment and the first bone segment, and the first bone rod penetrates the first bone segment and the second bone segment. The second bone rod is arranged at the connecting end between the second bone segment and the third bone segment, and the second bone rod penetrates the second bone segment and the third bone segment.

More preferably, one end of the first bone segment is provided with a first perforation, both ends of the second bone segment are provided with a second perforation and a third perforation, respectively, one end of the third bone segment is provided with a fourth perforation. When the second bone segment is connected to the first bone segment, the second perforation is aligned with the first perforation; when the second bone segment is connected to the third bone segment, the third perforation is aligned with the fourth perforation.

Preferably, the length of the part of the first tongue protruding from the first shoulder is equal to the depth of the first slot.

Preferably, the part of the first tongue protruding from the first shoulder is a dovetail.

The second aspect of the present disclosure provides a bone reconstruction connection method based on tenon-and-mortise fixation, which includes detachably connecting the adjacent bone segments through the first connecting part and the second connecting part arranged at the ends of the adjacent bone segments, the adjacent bone segments being described in the bone reconstruction connection structure based on tenon-and-mortise fixation in the first aspect of the present disclosure. When the adjacent bone segments are connected, the connection is performed by matching the first tongue in the first connecting part with the first slot in the second connecting part.

Preferably, the bone segments include a first bone segment, a second bone segment and a third bone segment connected in sequence. The bone reconstruction connection method includes: detachably connecting the first bone segment and the second bone segment through the first connecting part and the second connecting part arranged at the ends of the first bone segment and second bone segment, and when the first bone segment is connected to the second bone segment, the connection is performed by matching the first tongue in the first connecting part with the first slot in the second connecting part; detachably connecting the second bone segment and the third bone segment through the first connecting part and the second connecting part arranged at the ends of the second bone segment and the third bone segment, and when the second bone segment is connected to the third bone segment, the connection is performed by matching the first tongue in the first connecting part with the first slot in the second connecting part.

Preferably, the first bone segment and the second bone segment are obliquely connected to form a first bending angle of 60-180°; the second bone segment and the third bone segment are obliquely connected to form a second bending angle b of 60-180°. Among them, the bending directions of the first bone segment and the third bone segment relative to the second bone segment are opposite.

Preferably, the first bone rod penetrates the connecting end between the first bone segment and the second bone segment in sequence through the first perforation and the second perforation, the first bone rod is arranged at the connecting end between the second bone segment and the first bone segment, the first perforation is arranged at one end of the first bone segment, the second perforation is arranged at one end of the second bone segment; the second bone rod penetrates the connecting end between the second bone segment and the third bone segment in sequence through the third perforation and the fourth perforation, the second bone rod is arranged at the connecting end between the second bone segment and the third bone segment, the third perforation is arranged at another end of the second bone segment, and the forth perforation is arranged at one end of the third bone segment.

The third aspect of the present disclosure provides a method for promoting the healing of a bone broken end, including applying the bone reconstruction connection structure based on tenon-and-mortise fixation described in the first aspect to patients in need.

Preferably, the bone broken end is a mandible bone broken end. As described above, the present disclosure provides a bone reconstruction connection structure and method based on tenon-and-mortise fixation, which has the following beneficial effects:

(1) The bone reconstruction connection structure and method based on tenon-and-mortise fixation provided by the present disclosure, is only reinforced by its own tenon-and-mortise structure without the help of foreign objects such as fixing plate, thereby reducing the risks of infection, rejection and even secondary operation.

(2) The bone reconstruction connection structure and method based on tenon-and-mortise fixation provided by the present disclosure enable the biological force of the mandible to be effectively transmitted along the dental force track and muscle strength track through its own tenon-and-mortise joint structure. Further reinforcement by the bone rods can completely maintain the stability of the reconstructed bone segments and prevent dislocation, which is superior to the titanium plate fixing method that is not conducive to the conduction of mandibular biological force.

(3) The bone reconstruction connection structure and method based on tenon-and-mortise fixation provided by the present disclosure can ensure that each bone segment receives effective biomechanical stimulation while maintaining stability, thereby promoting bone fracture healing, and avoiding the problem that the bone segments do not receive effective stimulations of the biological force due to the titanium plate fixation.

(4) The bone reconstruction connection structure and method based on tenon-and-mortise fixation provided by the present disclosure are ingenious in design, simple in structure, convenient to use, low in cost, free of additional cost, and worthy of popularization and application in actual work.

### BRIEF DESCRIPTION OF THE DRAWINGS

FIG. 1 shows a schematic diagram of the fixed structure using a titanium plate rigid internal fixation method.

FIG. 2 shows a schematic diagram of the structure of the human mandible, and the track of dental force and muscle strength.

FIG. 3 shows a schematic diagram of the bone reconstruction connection structure based on tenon-and-mortise fixation at a connecting state according to the present disclosure.

FIG. 4 shows a schematic structural diagram of the bone reconstruction connection structure based on tenon-and-mortise fixation at a disassembled state according to the present disclosure.

FIG. 5 shows a schematic diagram of two different fixation methods of mandibular reconstruction with fibular flap. A: traditional titanium plate fixation; B: tenon-and-mortise fixation.

FIG. 6 shows a schematic diagram of the stress distribution of internal fixation of mandibular reconstruction with fibular flap based on two different fixation. A: traditional titanium plate fixation (after bone healing). B: traditional titanium plate fixation (before bone healing). C: tenon-and-mortise fixation (after bone healing). D: tenon-and-mortise fixation (before bone healing).

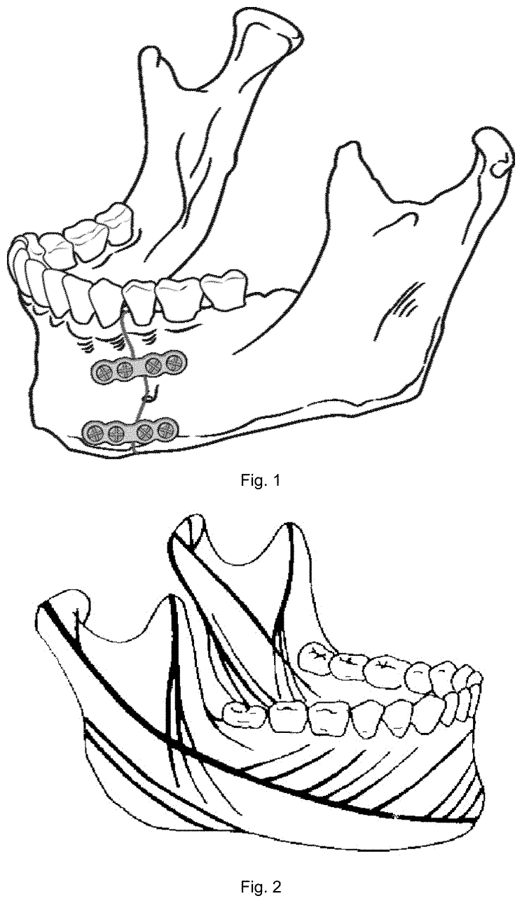

### REFERENCE NUMERALS

1 Bone segments
11 First bone segment
12 Second bone segment
13 Third bone segment
2 First connecting part
21 First tongue
22 First shoulder
3 Second connecting part
31 First slot
32 First slot shoulder
41 First bone rod
42 Second bone rod
51 First perforation
52 Second perforation
53 Third perforation
54 Fourth perforation
a First bending angle
b Second bending angle
c Muscle strength track
d dental force track

### DETAILED DESCRIPTION OF THE PREFERRED EMBODIMENTS

The following specific embodiments illustrate the implementation of the present disclosure. Those familiar skilled in the art can easily understand the other advantages and effects of the present disclosure from the content disclosed in this specification.

See FIGS. 2-4, it should be noted that the structure, ratio, size, etc. shown in the drawings in this specification are only used to match the content disclosed in the specification for the understanding and reading of those skilled in the art, and are not intended to limit the implementation of the present disclosure, therefore having no technical significance. Any structural modification, proportional relationship change or size adjustment should still fall within the scope of the present disclosure without affecting the effects and objectives that can be achieved by the present disclosure. At the same time, the terms such as “upper”, “lower”, “left”, “right”, “middle” and “one” quoted in this specification are only for the convenience of description and are not used to limit the text, the change or adjustment of its relative relationship, shall be regarded as falling within the scope of implementation of the present disclosure without substantial changes to the technical content.

### Embodiment 1 Bone Reconstruction Connection Structure Based on Tenon-and-Mortise Fixation

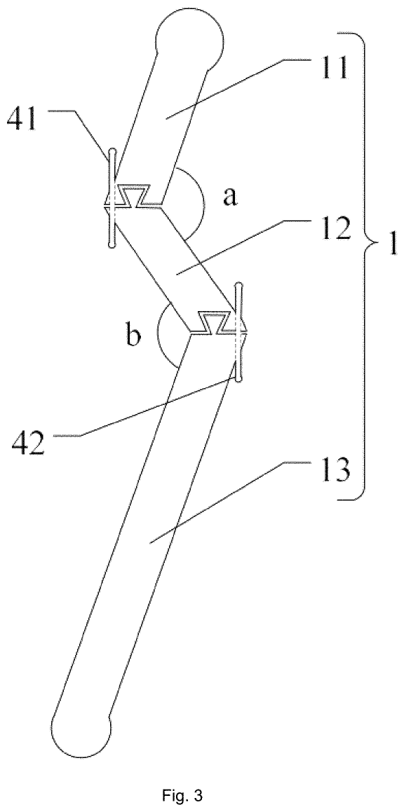

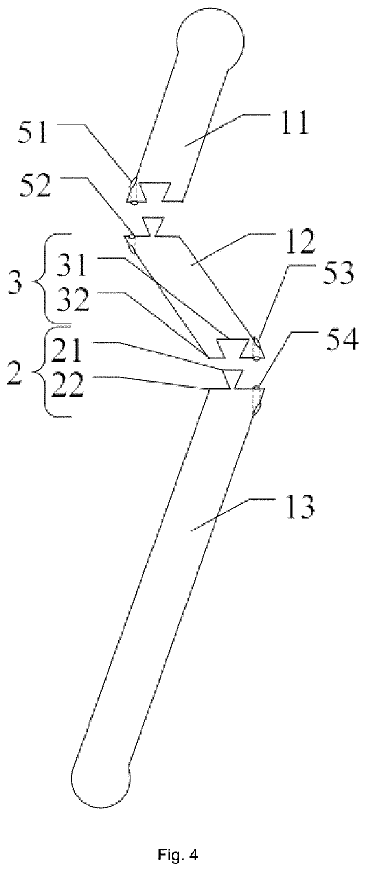

The present disclosure provides a bone reconstruction connection structure based on tenon-and-mortise fixation, as shown in FIGS. 3-4, including at least two bone segments 1, and the adjacent bone segments 1 are detachably connected through a first connecting part 2 and a second connecting part 3; the first connecting part 2 includes a first tongue 21 and a first shoulder 22, the first tongue 21 protrudes from the first shoulder 22, the second connecting part 3 includes a first slot 31 and a first slot shoulder 32, the first slot 31 is retracted relative to the first slot shoulder 32, and the part of the first tongue 21 protruding from the first shoulder 22 matches the first slot 31. The above-mentioned bone reconstruction connection structure based on tenon-and-mortise fixation is a specially designed connection part structure that can be tightly mounted to prevent vertical dislocation without the use of foreign objects such as fixation plates.

In a preferred embodiment, as shown in FIGS. 3-4, the bone segments 1 include a first bone segment 11, a second bone segment 12 and a third bone segment 13 connected in sequence. A first connecting part 2 or a second connecting part 3 arranged at one end of the second bone segment 12 is detachably connected to a second connecting part 3 or a first connecting part 2 arranged at one end of the first bone segment 11. A first connecting part 2 or a second connecting part 3 arranged at the other end of the second bone segment 12 is detachably connected to a second connecting part 3 or a first connecting part 2 arranged at one end of the third bone segment 13.

In a further preferred embodiment, as shown in FIG. 3, a first bending angle a of 60-180° is formed between the second bone segment 12 and the first bone segment 11; a second bending angle b of 60-180° is formed between the second bone segment 12 and the third bone segment 13; the bending directions of the first bone segment 11 and the third bone segment 13 relative to the second bone segment 12 are opposite.

The first bending angle a formed between one end of the second bone segment 12 and one end of the first bone segment 11 enables the first bone segment 11 and the second bone segment 12 to have a great retention force along the muscle strength track c as shown in FIG. 2, to prevent dislocation. The second bending angle b formed between the other end of the second bone segment 12 and one end of the third bone segment 13 enables the second bone segment 12 and the third bone segment 13 to have a great retention force along the dental force track d as shown in FIG. 2, to prevent dislocation.

In a further preferred embodiment, as shown in FIG. 3, the connecting end between the second bone segment 12 and the first bone segment 11, and the connecting end between the second bone segment 12 and the third bone segment 13 are provided with bone rods, and the bone rods include a first bone rod 41 and a second bone rod 42. The first bone rod 41 is arranged at the connecting end between the second bone segment 12 and the first bone segment 11, and the first bone rod 41 penetrates the first bone segment 11 and the second bone segment 12. The second bone rod 42 is arranged at the connecting end between the second bone segment 12 and the third bone segment 13, and the second bone rod 42 penetrates the second bone segment 12 and the third bone segment 13.

The first bone rod 41 can further strengthen and maintain the stability of the reconstructed bone segment between the second bone segment 12 and the first bone segment 11, and prevent the second bone segment 12 and the first bone segment 11 from being dislocated in a vertical direction. The second bone rod 42 can further strengthen and maintain the stability of the reconstructed bone segment between the second bone segment 12 and the third bone segment 13, and prevent the second bone segment 12 and the third bone segment 13 from being dislocated in a vertical direction.

Specifically, as shown in FIG. 3, the bone rod has a cylindrical shape and can fix the bone segment 1 without falling off. In a further preferred embodiment, as shown in FIG. 4, one end of the first bone segment 11 is provided with a first perforation 51; both ends of the second bone segment 12 are provided with a second perforation 52 and a third perforation 53, respectively; one end of the third bone segment 13 is provided with a fourth perforation 54. When the second bone segment 12 is connected to the first bone segment 11, the second perforation 52 is aligned with the first perforation 51; when the second bone segment 12 is connected to the third bone segment 13, the third perforation 53 is aligned with the fourth perforation 54.

After the second perforation 52 is aligned with the first perforation 51, it is convenient for the first bone rod 41 to penetrate the first bone segment 11 and the second bone segment 12. After the third perforation 53 is aligned with the fourth perforation 54, it is convenient for the second bone rod 42 to penetrate the second bone segment 12 and the third bone segment 13.

In a preferred embodiment, the material of the bone segment 1 may be human bones. In a preferred embodiment, the first bone segment 11 and third bone segment 13 are mandible, and the second bone segment 12 is fibula. When the bone segment 1 is a human bone, the osteotomy and drilling operation of the bone segment 1 may be realized by a power saw, a drill or other osteotomy devices. Personalized osteotomies may be precisely implemented with surgical guides, optical navigation and robotic equipment.

The part of the first tongue 21 protruding from the first shoulder 22 matches the first slot 31, meaning that the part of the first tongue 21 protruding from the first shoulder 22 can be completely inserted into the first slot 31, and the two coincide to form a tightly connected tenon-and-mortise structure.

In a preferred embodiment, as shown in FIG. 4, the length of the part of the first tongue 21 protruding from the first shoulder 22 is equal to the depth of the first slot 31.

In a preferred embodiment, as shown in FIG. 4, the part of the first tongue 21 protruding from the first shoulder 22 is a dovetail. Specifically, the part of the first tongue 21 protruding from the first shoulder 22 is in a shape of a square frustum, and one end surface of the part of the first tongue 21 protruding from the first shoulder 22 is connected to the bone segment 1. The areas between the other end surface of the part of the first tongue 21 protruding from the first shoulder 22 and the connection surface of the bone segment 1 decrease gradually as it gets close to the connection surface of the bone segment 1. At the same time, the first slot 31 matching the part of the first tongue 21 protruding from the first shoulder 22 opens on an one end surface and both sides. The first slot 31 has a shape of a square frustum, the areas between the opening end surface of the first slot 31 and an end surface connecting with the retraction part of the first slot shoulder 32 gradually decreases.

### Embodiment 2 Bone Reconstruction Connection Method Based on Tenon-and-Mortise Fixation

The following describes a bone reconstruction connection method based on tenon-and-mortise fixation in the present disclosure with reference to FIGS. 2-4.

After the user obtains a bone reconstruction connection structure based on tenon-and-mortise fixation as shown in FIGS. 2-4, the bone reconstruction connection structure based on tenon-and-mortise fixation in the present disclosure is used for the patient's mandible fracture which needs to be reconstructed.

Firstly, detachably connect the first bone segment 11 and the second bone segment 12, through the first connecting part 2 and the second connecting part 3 arranged at the ends of the first bone segment 11 and the second bone segment 12. The first bone segment 11 and the second bone segment 12 are obliquely connected to form a first bending angle a of 60-180°. When the first bone segment 11 and the second bone segment 12 are connected, the connection is performed by matching the first tongue 21 in the first connecting part 2 with the first slot 31 in the second connecting part 3. Since the part of the first tongue 21 protruding from the first shoulder 22 is a dovetail, and matches the shape of the first slot 31, it can be effectively connected to the first slot 31. To strengthen and maintain the stability of the reconstructed bone segment between the second bone segment 12 and the first bone segment 11, and to prevent the second bone segment 12 and the first bone segment 11 from being dislocated in a vertical direction, the first bone rod 41 penetrates the connecting end between the first bone segment 11 and the second bone segment 12 in sequence through the first perforation 51 and the second perforation 52, thereby enabling the first bone segment 11 and the second bone segment 12 to have a great retention force along the muscle strength track c and to prevent dislocation.

Secondly, detachably connect the second bone segment 12 and the third bone segment 13, through the first connecting part 2 and the second connecting part 3 arranged at the ends of the second bone segment 12 and the third bone segment 13. The second bone segment 12 and the third bone segment 13 are obliquely connected to form a second bending angle b of 60-180°. When the second bone segment 12 and the third bone segment 13 are connected, the connection is performed by matching the first tongue 21 in the first connecting part 2 with the first slot 31 in the second connecting part 3. Since the part of the first tongue 21 protruding from the first shoulder 22 is a dovetail, and matches the shape of the first slot 31, it can be effectively connected to the first slot 31. To strengthen and maintain the stability of the reconstructed bone segment between the second bone segment 12 and the third bone segment 13, and to prevent the second bone segment 12 and the third bone segment 11 from being dislocated in a vertical direction, the second bone rod 42 penetrates the connecting end between the second bone segment 12 and the third bone segment 13 in sequence through the third perforation 53 and the forth perforation 54, thereby enabling the second bone segment 12 and the third bone segment 13 to have a great retention force along the dental force track d and to prevent dislocation.

In summary, the present disclosure provides a bone reconstruction connection structure and method based on tenon-and-mortise fixation, which only uses its own tenon-and-mortise structure for reinforcement, thereby reducing the risks such as infection, rejection and even secondary operations caused by implant fixation, promoting the healing of bone fractures. Therefore, the present disclosure effectively overcomes various shortcomings and has a high industrial value.

### Embodiment 3 Simulation of Stress Distribution and Stress Peak of the Internal Fixators for Bone Reconstruction Connection Methods Based on Tenon-and-Mortise Fixation and Traditional Titanium Plate Fixation

The stress peak of the internal fixator often determines the probability of fracture or deformation of the internal fixator. The higher the stress peak, the higher the risk of fracture or deformation. In other words, a fixation method with a low stress peak of its internal fixator is safer than those with high stress peak of their internal fixators.

Therefore, in this Embodiment, three-dimensional finite element analysis was carried out to investigate the safety of the tenon-and-mortise fixation of the present disclosure.

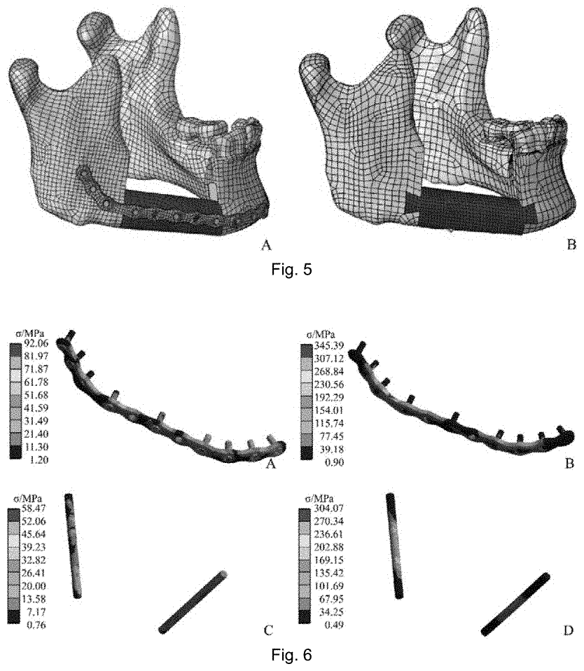

Imaging data of the mandible and fibula of a patient (66-year old, having no fracture or deformity in the mandible) were obtained by CT scan. Three-dimensional reconstruction and reverse reconstruction were performed by software Mimics 17.0 and Geomagic Wrap 2017 to obtain the three-dimensional model of the mandible and fibula. The final model of mandibular reconstruction with a fibular flap was obtained by software Solidworks 2017 and Geomagic Design X64, as shown in FIG. 5. Traditional titanium plate fixation and tenon-and-mortise fixation were both simulated. In the tenon-and-mortise fixation model of the present Embodiment, two ends of the fibula are cut into a hexahedral shape, so that the fibula serves as a tenon. A notch is cut at the mandible and serves as a mortise, to match the hexahedral shape of the ends of the fibula. The mortise and tenon are joined.

Three-dimensional finite element analysis was used to compare and analyze the stress distribution of the internal fixators before and after bone healing with different fixation modes (tenon-and-mortise fixation; traditional titanium plate).

Material parameters for finite element model analysis can be found in Table 1.

TABLE 1
Material parameters for finite element model analysis

| | Elasticity modulus | |
|---|---|---|
| Material | (MPa) | Poisson's ratio |
| Cortical bone of the mandible | 13700 | 0.3 |
| Cancellous bone of the mandible | 7930 | 0.3 |
| Fibula | 13700 | 0.3 |
| Titanium | 110000 | 0.3 |

The stress distribution and stress peaks of internal fixators of the tenon-and-mortise fixation method and traditional titanium plate fixation method are obtained by simulating a vertical occlusal state (loading conditions: 125N downward along the axis of the central incisor, and 250N downward along the axis of the second and third molars).

Results of stress distribution and stress peak are shown in FIG. 6. In detail, before bone healing, the stress peak of the internal fixator fixed by tenon-and-mortise structure was distributed at the junction of bone section and internal fixator, which was 304.07 MPa; the peak stress of the titanium plate fixation was 345.39 MPa at the broken end of the posterior bone. After bone healing, the stress distribution of the internal fixator fixed by tenon-and-mortise structure remained unchanged, and the stress peak was 58.47 MPa; the stress peak of the internal fixator fixed by titanium plate fixation was concentrated at the contact point between the titanium nail and the titanium plate, which was 92.06 MPa.

The above results show that the stress peaks of the internal fixator of the tenon-and-mortise fixation, both before bone healing and after bone healing, are lower than those of the traditional titanium plate fixation, indicating a lower probability of fracture or deformation. Therefore, the tenon-and-mortise fixation of the present disclosure is safer for the reconstruction of the mandible with fibular flap than traditional titanium plate fixation.

The above-mentioned embodiments only exemplarily illustrate the principles and effects of the present disclosure, but are not used to limit the present disclosure. Anyone familiar with this technology can modify or change the above-mentioned embodiments without departing from the spirit and scope of the present disclosure. Therefore, all equivalent modifications or changes made by those skilled in the art without departing from the spirit and technical ideas disclosed in the present disclosure should still be covered by the claims of the present disclosure

What is claimed is:

1. A bone reconstruction connection structure based on tenon-and-mortise fixation, comprising at least two bone segments (1), and adjacent bone segments (1) are detachably connected through a first connecting part (2) and a second connecting part (3); the first connecting part (2) comprises a first tongue (21) and a first shoulder (22), the first tongue (21) protrudes from the first shoulder (22), the second connecting part (3) comprises a first slot (31) and a first slot shoulder (32), the first slot (31) is retracted relative to the first slot shoulder (32), and a part of the first tongue (21) protruding from the first shoulder (22) matches the first slot (31).

2. The bone reconstruction connection structure based on tenon-and-mortise fixation according to claim 1, wherein the bone segments (1) comprise a first bone segment (11), a second bone segment (12) and a third bone segment (13) connected in sequence, a first connecting part (2) or a second connecting part (3) arranged at one end of the second bone segment (12) is detachably connected to a second connecting part (3) or a first connecting part (2) arranged at one end of the first bone segment (11), a first connecting part (2) or a second connecting part (3) arranged at the other end of the second bone segment (12) is detachably connected to a second connecting part (3) or a first connecting part (2) arranged at one end of the third bone segment (13).

3. The bone reconstruction connection structure based on tenon-and-mortise fixation according to claim 2, wherein a first bending angle a of 60-180° is formed between the second bone segment (12) and the first bone segment (11); a second bending angle b of 60-180° is formed between the second bone segment (12) and the third bone segment (13); the bending directions of the first bone segment (11) and the third bone segment (13) relative to the second bone segment (12) are opposite.

4. The bone reconstruction connection structure based on tenon-and-mortise fixation according to claim 2, wherein a connecting end between the second bone segment (12) and the first bone segment (11), and a connecting end between the second bone segment (12) and the third bone segment (13) are provided with bone rods, and the bone rods comprise a first bone rod (41) and a second bone rod (42); the first bone rod (41) is arranged at the connecting end between the second bone segment (12) and the first bone segment (11), and the first bone rod (41) penetrates the first bone segment (11) and the second bone segment (12); the second bone rod (42) is arranged at the connecting end between the second bone segment (12) and the third bone segment (13), and the second bone rod penetrates the second bone segment (12) and the third bone segment (13).

5. The bone reconstruction connection structure based on tenon-and-mortise fixation according to claim 2, wherein one end of the first bone segment (11) is provided with a first perforation (51), both ends of the second bone segment are provided with a second perforation (52) and a third perforation (53), respectively, one end of the third bone segment is provided with a fourth perforation (54), when the second bone segment (12) is connected to the first bone segment (11), the second perforation (52) is aligned with the first perforation (51); when the second bone segment (12) is connected to the third bone segment (13), the third perforation (53) is aligned with the fourth perforation (54).

6. The bone reconstruction connection structure based on tenon-and-mortise fixation according to claim 1, wherein the part of the first tongue (21) protruding from the first shoulder (22) is a dovetail.

7. A bone reconstruction connection method based on tenon-and-mortise fixation, comprising:
providing a bone reconstruction connection structure based on tenon-and-mortise fixation according to claim 1; and
detachably connecting the adjacent bone segments (1) through the first connecting part (2) and the second connecting part (3) arranged at the ends of the adjacent bone segments (1), when the adjacent bone segments (1) are connected, the connection is performed by matching the first tongue (21) in the first connecting part (2) with the first slot (31) in the second connecting part (3).

8. The bone reconstruction connection method based on tenon-and-mortise fixation according to claim 7, wherein the bone segments (1) comprise a first bone segment (11), a second bone segment (12) and a third bone segment (13) connected in sequence, the bone reconstruction connection method comprises:
detachably connecting the first bone segment (11) and the second bone segment (12) through the first connecting part (2) and the second connecting part (3) arranged at the ends of the first bone segment (11) and the second bone segment (12), wherein when the first bone segment (11) is connected to the second bone segment (12), the connection is performed by matching the first tongue (21) in the first connecting part (2) with the first slot (31) in the second connecting part (3); and
detachably connecting the second bone segment (12) and the third bone segment (13) through the first connecting part (2) and the second connecting part (3) arranged at the ends of the two, wherein when the second bone segment (12) is connected to the third bone segment (13), the connection is performed by matching the first tongue (21) in the first connecting part (2) with the first slot (31) in the second connecting part (3).

9. The bone reconstruction connection method based on tenon-and-mortise fixation according to claim 8, wherein:
the first bone segment (11) and the second bone segment (12) are obliquely connected to form a first bending angle a of 60-180°;
the second bone segment (12) and the third bone segment (13) are obliquely connected to form a second bending angle b of 60-180°;
wherein bending directions of the first bone segment (11) and the third bone segment (13) relative to the second bone segment (12) are opposite.

10. The bone reconstruction connection method based on tenon-and-mortise fixation according to claim 8, further comprising:
penetrating, via a first bone rod (41), a connecting end between the first bone segment (11) and the second bone segment (12) in sequence through a first perforation (51) and a second perforation (52), wherein the first bone rod (41) is arranged at the connecting end between the second bone segment (12) and the first bone segment (11), the first perforation (51) is arranged at one end of the first bone segment (11), and the second perforation (52) is arranged at one end of the second bone segment (12);
penetrating, via the second bone rod (42), a connecting end between the second bone segment (12) and the third bone segment (13) in sequence through a third perforation (53) and a fourth perforation (54), wherein the second bone rod (42) is arranged at the connecting end between the second bone segment (12) and the third bone segment (13), the third perforation (53) is arranged at another end of the second bone segment (12), and the forth perforation (54) is arranged at one end of the third bone segment (13).

11. A method for promoting the healing of a bone broken end, comprising: applying the bone reconstruction connection structure based on tenon-and-mortise fixation according to claim 1 to patients in need.
